(19) United States
(12) Patent Application Publication
(10) Pub. No.: US 2025/0276171 A1
(43) Pub. Date: Sep. 4, 2025

(54) COLLAPSIBLE IMPELLER WIRE FRAMES FOR BLOOD PUMP

(71) Applicant: VENSTRAMEDICAL PTY LTD, Newcastle (AU)

(72) Inventors: Martin Christopher Cook, Eden Prairie, MN (US); Sukumaran Thevathasan Thambar, Bar Beach, New South Wales (AU); David Michael Heisserer, Minneapolis, MN (US); Nathaniel Zenz-Olson, Ham Lake, MN (US); Karl A. Kabarowski, Plymouth, MN (US)

(21) Appl. No.: 18/854,510

(22) PCT Filed: Apr. 19, 2023
(86) PCT No.: PCT/US2023/019123

§ 371 Date: Oct. 4, 2024

Related U.S. Application Data
(60) Provisional application No. 63/363,314, filed on Apr. 20, 2022.

(51) Int. Cl. A61M 60/808; A61M 60/13; A61M 60/237

(52) U.S. Cl. CPC A61M 60/808; A61M 60/13; A61M 60/237

(57) ABSTRACT

A radially compressible and expandable rotor for a blood pump can include a rotor comprising at least one impeller blade having a flexible blade body, wherein the at least one impeller blade includes an elastically deformable first material that is supported by a relatively stiffer second material of a structural frame encapsulated within the blade body. The relatively stiffer second material of the structural frame can be adapted to give a supporting structure to the impeller blade body, wherein when expanded, the structural frame has a predetermined expanded shape. The second material of the structural frame can have a shape with a symmetry along a. longitudinal axis defined by the rotor.

### CLAIM OF PRIORITY

This patent application claims the benefit of priority of U.S. Provisional Patent Application Ser. No. 63/363,314, entitled “Wire Frames within a Collapsible Impeller for a Blood Pump,” filed on Apr. 20, 2022, which is hereby incorporated by reference herein in its entirety.

### DETAILED DESCRIPTION

In the following description, for purposes of explanation, various details are set forth in order to provide a thorough understanding of some example embodiments. It will be apparent, however, to one skilled in the art that the present subject matter may be practiced without these specific details, or with slight alterations.

### TECHNICAL FIELD

The present invention relates to wire frames within a collapsible impeller for a blood pump and more specifically, to a radially compressible and expandable rotor having an impeller with a structural frame encapsulated within the impeller body.

### BACKGROUND

Mechanical circulatory support has become a standard of practice for the treatment of late-stage heart failure. The most common method of providing mechanical circulatory support is a left ventricular assist device (“LVAD”), which is a pump that takes over much of, if not all, the function of the left ventricle. LVADs are larger devices that may be placed via a surgical implantation technique for chronic support. In contrast, an intracardiac blood pump is a smaller device that can be implanted into the heart without major surgery via a catheter delivered through the arterial/venous system. Such devices are often called ‘percutaneous pumps’ or ‘catheter pumps’.

Percutaneous pump housings must have a relatively small diameter to allow for positioning via the vascular system of the patient and are typically less than about 7 mm in diameter. Percutaneous pumps designed to support a failing left ventricle generally provide a blood flow output of about 4.5 L/min against 60-80 mmHg pressure, although lower flows may be acceptable for partial support.

Percutaneous pumps are usually designed to be placed or positioned during use such that they are disposed within or across the aortic valve. As such, these pumps are made very small and also that they can be compressed for transport and expanded for operation. This specific application of such pumps in the medical field relates to invasive blood pumps which can be conveyed into the body of a patient in the radially compressed state and upon activation, can be expanded there to pump blood.

U.S. Pat. No. 7,393,181 discloses a rotor with a plurality of vanes which are manufactured in one piece with a hub there and can be folded onto the hub in the compressed state due to their material elasticity in order to compress the rotor radially. In operation, the vanes erect themselves. While the material of the rotor has to be selected carefully with respect to its elasticity properties and deformability properties, there is a long felt need to be have a fine balance between the impeller being flexible to fold and also be able to exert sufficient force on the liquid or blood to be conveyed in operation.

Other radially compressible and expandable rotors for a blood pump having an impeller blade is known in U.S. Pat. No. 10,920,596 B2 which has support structures that does not have any closed edge structure or edge curve, in which the stiffening strut is at least partially embedded in the material of the impeller blade body and the stiffening strut has stiffening curvature which is preferably in the region radially close to the hub.

Similar pumps are known from US Patent Application Publication No. US2008/0114339A1 and U.S. Pat. No. 9,611,743 B2, in which the radially compressible and expandable rotor for a pump with a stiffening strut which is at least partially embedded in the material of the impeller blade body, wherein the impeller blade thickness between the pressure side and the suction side amounts to at least 80% of the thickness of the at least one strut in the same direction.

Any discussion of the prior art throughout the specification should in no way be considered as an admission that such prior art is widely known or forms part of common general knowledge in the field. The present invention relates to wire frames within a collapsible impeller for a blood pump and more specifically, to a radially compressible and expandable rotor having an impeller with a structural frame encapsulated within the impeller body.

### SUMMARY

The present inventors have recognized, among other things, that a problem to be solved can include the location and deployment of a percutaneous device within the vasculature of a patient to support blood flow within the patient. The present subject matter can help provide a solution to this problem, such as an expandable and collapsible impeller with an embedded structural frame, such as configured for use in a percutaneous blood pump.

It may be an advantage to provide a radially compressible and expandable rotor for a blood pump having an impeller blade with a structural frame encapsulated by the flexible impeller blade body for providing functional support and shape, when in operation.

It may be an advantage to provide a structural frame having a skeletal shape chosen from the group of: honeycomb cell, diamond cell, rectangular cell, dragonfly wing, wavy strut wing, diagonal strut wing. These shapes may provide even support to the flexible impeller blade body.

It may be advantageous to provide a device for assisting blood flow from the left ventricle to the aorta of the heart with the blood being expelled from the device at the left ventricle in the direction of the atrioventricular valve.

It may be an advantage to provide a device with a collapsible impeller and a collapsible impeller cage to keep the size the device as small as possible and also reducing the risk of snagging when inserting and moving the device through the tortuous anatomy of the heart to the desired location. Collapsible configuration of the pump and impeller allows for easier insertion or implantation of the device via the femoral artery reducing the need for sternotomy.

It may be advantage to provide the collapsible impeller and a collapsible impeller cage with a shape memory metal so that it retains its functional shape to operate when the impeller and the impeller cage are expanded at the desired location.

It may be an advantage to provide a structural frame having a honeycomb pattern so as to resist stretch and compression in the helical plane that it is oriented. When that plane follows the helical shape, compound curves can be created. Those compound curves, with their planar stability become very resistant to deform the overall structure. While at the same time, the voids in the plane allow us to forcefully overcome the heatset structure temporarily. The displacement of polymer, required to collapse the impeller blade is also aided by the cellular nature and regularity of the honeycomb structure.

It may be an advantage to provide a structural frame having diamond patterns so as to have an economical way to make planar shapes with less material. Diamond structures in a plane can have differing resistance to stretch and compression along different axes. The advantage of having the structural frame having diamond patterns with the orientation within the helical plane to utilize compressibility or expandability where necessary to aid in the collapse of the impeller blade, such as a helically planar impeller blade. Long struts comprised of numerous edges of individual diamonds could be utilized to create strength by directly spanning longer distances, for a given application, which is a trait less available in a honeycomb pattern. The displacement of polymer, required to collapse the impeller blade may also be aided by the cellular nature and regularity of the diamond structure.

It may be an advantage to have less metal at the trailing edge and the metal frame may be tapered away from the trailing edge. So that the trailing edge is more easily folded or collapsed as the sheath is advanced from the trailing edge. Once the trailing edge starts to collapse, it may make it easier for the rest of the rotor and the impeller body to also fold.

It may be an advantage to provide a structural frame that is included to allow for the impeller blade that will deform less when under use and pressure forces are working to bend the impeller.

It may be an advantage to provide a blood pump that is delivered via the vascular system to be placed in the left ventricle, in which this blood pump is implantable and typically require no surgery. For which this blood pump is used during high-risk coronary interventions (such as stent replacement) when the heart may cease to provide flow and this blood pump is urgently needed to provide cardiac support during the procedure. It may be a further advantage to provide this blood pump to be used for patients experiencing cardiogenic shock which is when a patient is suffering severe cardiac insufficiency and is at a life-threatening condition.

It may be an advantage to allow for an impeller blade that will deform less when under use and pressure forces are working to bend the rotor. The metal frame may work to oppose this hence the impeller blade may be able to maintain their shape and hence enhance hydraulic (hemodynamic) performance and minimize blood damage (hemolysis)

It may be an advantage to provide a metal frame that is preferably laser cut from a flat sheet of super elastic nitinol to allow it to fold tightly to a small diameter and then rebound to the design shape when the sheath is removed. The frame may then be ‘heatset’ to the desired shape before the polymer is added to encapsulate the frame.

It may be an advantage to allow the polymer that encapsulates the frame to mechanically lock into all the ‘elements’ of the frame (such as ‘diamond’, ‘honeycomb’ etc) which may ensure a good adherence to the frame and may prevent detachment or delamination which may be an issue otherwise.

It may be an advantage to provide a wire frame that can be made thinly (approximately 0.13 mm) and can be electropolished to round edges and prevent the frame ‘cutting’ through the polymer.

It may be an advantage to provide an approach that may allow for injection molding as well as casting or vacuum casting. In an example, a first material, such as an elastically deformable material, can include a polymer. Polymer materials are preferrable polyurethanes with known hemocompatibility, such as a material from Lubrizol (Wickliffe, Ohio) offered for sale under the trademark PELLETHANE® or another material from Lubrizol (Wickliffe, Ohio) offered for sale under the trademark TECOFLEX®, but can also be silicones or other hemocompatible polymers.

It may be an advantage to provide various ways of joining the wire frame to the rotor or shaft such as rotor traversing portions that wrap around the rotor or shaft, or a rotor with wire frame locking means or the wire frame welded to the rotor.

It may be an advantage to provide a wire frame that is more dense or more wire frames close to the leading edge where the pressure forces on the impeller blades are greater. This is so that the impeller body can be tailored by the wire frame to provide most support where it is needed and less where is not needed thus allowing those sections to be more flexible and collapse with less force.

It may be an advantage to allow laser cutting from sheet with the nitinol frame approach regardless of the different wire frame designs which will allow a thinner impeller blade overall and therefore can collapse into a smaller catheter.

It may be an advantage to provide a structural frame, such as made from at least one of a polymer material or a metal material, such as an alloy of nickel titanium (e.g., nitinol). In an example, the structural frame can assume a predetermined expanded shape, such as the structural frame can assume the predetermined shape after transition from a compressed state, such as for percutaneous insertion into a patient, to an expanded state, such as in preparation for operation within the patient vasculature.

It may be an advantage to provide a wire frame that may not be metal but could be an elastic material other than nitinol that has the same shape retaining properties as nitinol.

The structural frame can include a wire frame. In an example, the impeller blade can be constructed from a first material, such as an elastically deformable first material, and a second material, such as a structural frame made from a material that is relatively stiffer than the first material. The structural frame, such as the structural frame made from a second material, can be encapsulated in a first material, such as the elastically deformable first material, to form an impeller blade. The encapsulated structural frame can provide a supporting structure to the impeller blade body.

It may be an advantage to provide a structural frame that is symmetric, such as about an axis of the impeller. In an example, the structural frame can display symmetry about a longitudinal axis, such as the longitudinal axis of the rotor. In an example, the term impeller can be used as a synonym for the term rotor.

It is an object of the present invention to overcome or ameliorate at least one of the disadvantages of the prior art, or to provide a useful alternative.

### MEANS FOR SOLVING THE PROBLEM

A first aspect of the present invention may relate to a radially compressible and expandable rotor for a blood pump, the rotor comprising: at least one impeller blade having a flexible blade body, wherein the at least one impeller blade includes a structural frame encapsulated within the blade body; wherein the structural frame is adapted to give structure to the impeller blade body, wherein when expanded, the structural frame has a predetermined expanded shape.

Preferably, the structural frame is attached to an outer surface of the rotor. In an example, the structural frame is attached to an outer surface of a longitudinally elongate rotor hub of the rotor extending along the longitudinal axis.

Preferably, the structural frame comprises one or more rotor traversing portions, wherein each of the rotor traversing portions traverses the rotor perpendicularly relative to the longitudinal axis of the rotor.

Preferably, the rotor has one or more bores extending perpendicular to the longitudinal axis of the rotor.

Preferably, a first bore of the rotor is adapted for receiving a first rotor traversing portion.

Preferably, a second bore of the rotor is adapted for receiving a second rotor traversing portion, wherein the second bore is adjacent to the first bore relative to the longitudinal axis of the rotor.

Preferably, the structural frame is a ‘fishbone’ skeletal shape.

Preferably, the structural frame further comprises one or more traversing coiled portions, wherein when attached to the rotor, each of the coiled portions is respectively coiled between a wall of the bore and an outer surface of the rotor.

Preferably, the structural frame has an arcuate portion positioned between the first traversing portion and a second traversing portion.

Preferably, the structural frame is a ‘petal’ skeletal shape.

Preferably, the structural frame is self-centering within the flexible blade body, when expanded.

Preferably, the structural frame is made from a single piece of material.

Preferably, the structural frame comprises a rotor engagement frame, wherein the rotor engagement frame is adapted to secure the structural frame to the rotor.

Preferably, the structural frame comprises at least one skeletal shape chosen from the group of: ‘honeycomb’ cell, ‘diamond’ cell, ‘rectangular’ cell, a first type of ‘dragonfly’ wing, a second type of ‘dragonfly’ wing, ‘wavy’ strut wing, ‘diagonal’ strut wing.

Preferably, the structural frame comprises a rotor engagement frame, wherein the rotor engagement frame is adapted to secure the structural frame to the rotor.

Preferably, the rotor engagement frame has an upper frame and a lower frame, wherein the upper frame is parallel to the lower frame, and wherein the upper frame and the lower frame are each parallel to the longitudinal axis of the rotor.

Preferably, at least one rotor reinforcement frame is in connection between the upper frame and the lower frame, wherein the rotor reinforcement frame comprises a rotor engagement portion positioned between the upper and lower frames.

Preferably, a first rotor reinforcement portion is adapted to secure a first portion of the outer surface of the rotor.

Preferably, a second rotor reinforcement portion is adapted to secure a second portion of the outer surface of the rotor.

Preferably, the secured first portion and the secured second portion of the outer surface of the rotor is diametrically opposed from each other.

In the context of the present invention, the words “comprise”, “comprising” and the like are to be construed in their inclusive, as opposed to their exclusive, sense, that is in the sense of “including, but not limited to”.

The invention is to be interpreted with reference to the at least one of the technical problems described or affiliated with the background art. The present aims to solve or ameliorate at least one of the technical problems and this may result in one or more advantageous effects as defined by this specification and described in detail with reference to the preferred embodiments of the present invention.

Each of these non-limiting examples can stand on its own, or can be combined in various permutations or combinations with one or more of the other examples.

This overview is intended to provide an overview of subject matter of the present patent application. It is not intended to provide an exclusive or exhaustive explanation of the invention. The detailed description is included to provide further information about the present patent application.

### BRIEF DESCRIPTION OF THE DRAWINGS

In the drawings, which are not necessarily drawn to scale, like numerals may describe similar components in different views. Like numerals having different letter suffixes may represent different instances of similar components. The drawings illustrate generally, by way of example, but not by way of limitation, various embodiments discussed in the present document.

FIG. 1A illustrates a basic anatomy of the collapsible blood pump.

FIG. 1B illustrates a collapsible impeller and collapsible outer pump housing at the compressed state.

FIG. 1C illustrates the outer sheath being removed to start to expand the outer housing and impeller of FIG. 1B.

FIG. ID illustrates the deployed state with the collapsible impeller and collapsible outer pump housing expanded and in use in pumping blood.

FIG. 2A illustrates a collapsible impeller body having a wire in a helical shape in the polymer membrane.

FIG. 2B also illustrates a collapsible impeller body having a wire in a helical shape in the polymer membrane.

FIG. 3 illustrates a collapsible impeller body of FIG. 2A or FIG. 2B rotated 90° around the longitudinal axis of the rotor.

FIG. 4 illustrates a collapsible impeller body having a petal wire frame that involves a single (or multiple) individual semi-loop reinforcing members within the polymer collapsible rotor.

FIG. 5A illustrates a ‘fishbone’ wire frame having reduced loop density within the collapsible impeller body.

FIG. 5B illustrates another embodiment of a ‘fishbone’ wire frame having reduced loop density within the collapsible impeller body, wherein the ‘fishbone’ wire frame is self-centering.

FIGS. 5C to 5F illustrates different perspective views of another embodiment of a ‘fishbone’ wire frame within the collapsible impeller body.

FIG. 5G illustrates a top view or when viewed in line of the longitudinal axis of the collapsible rotor of any one of FIGS. 5C to 5F, where the ‘fishbone’ wire frame follows a ‘slalom’ configuration that reinforces the collapsible rotor.

FIG. 5H illustrates a side view of the collapsible impeller body wherein the ‘fishbone’ wire frame follows a ‘slalom’ configuration that reinforces the collapsible rotor.

FIG. 5I illustrates the shape of the ‘fishbone’ wire of FIG. 5H.

FIG. 5J illustrates the shape of the ‘fishbone’ wire of FIG. 5G.

FIG. 5K illustrates the shape of the ‘fishbone’ wire of FIG. 5F.

FIG. 6 illustrates another embodiment of a wire frame, wherein this particular design is a ‘wavy strut’ wire frame.

FIG. 7 illustrates a ‘honeycomb’ wire frame design used within the collapsible impeller body.

FIG. 8 illustrates a ‘diamond’ wire frame design used within the collapsible impeller body.

FIG. 9 illustrates a first type of ‘dragonfly’ wire frame design in engagement with a collapsible rotor.

FIG. 10 illustrates a second type of ‘dragonfly’ wire frame design within the collapsible impeller body.

FIG. 11 illustrates a ‘honeycomb’ wire frame design within the collapsible impeller body, in which both the leading edge and the trailing edge each extends perpendicular relative from the longitudinal axis of the rotor.

FIG. 12 illustrates a ‘honeycomb’ wire frame design within the collapsible impeller body, in which both the leading edge and the trailing edge each extends arcuately relative from the longitudinal axis of the rotor.

FIG. 13 illustrates another a ‘honeycomb’ wire frame design within another embodiment of the collapsible impeller body.

FIG. 14 is a representative diagram of an impeller body with a ‘honeycomb’ wire design within the collapsible impeller body, wherein the ‘honeycomb’ pattern will have the tendency to resist stretch and compression in the directions as indicated by the direction of the arrows illustrated on the impeller body.

FIG. 15 illustrates a second type of ‘dragonfly’ wire frame design within the collapsible impeller body, in which both the leading edge and the trailing edge each extends perpendicular relative from the longitudinal axis of the rotor.

FIG. 16 illustrates a second type of ‘dragonfly’ wire frame design within the collapsible impeller body, in which both the leading edge and the trailing edge each extends arcuately relative from the longitudinal axis of the rotor.

FIG. 17 illustrates a second type of ‘dragonfly’ wire frame design within another embodiment of the collapsible impeller body.

FIG. 18 illustrates a ‘diamond’ wire frame design within the collapsible impeller body, in which both the leading edge and the trailing edge each extends perpendicular relative from the longitudinal axis of the rotor.

FIG. 19 and FIG. 20 each illustrates a ‘diamond’ wire frame design within another embodiment of the collapsible impeller body.

FIG. 21 is a representative diagram of an impeller body with a ‘diamond’ wire design within the collapsible impeller body, wherein the ‘diamond’ pattern will have the tendency to resist stretch and compression in the directions as indicated by the direction of the arrows illustrated on the impeller body.

FIG. 22 illustrates a flat laser cut design (prior to shaping and overmolding) of another ‘diamond’ wire frame design.

FIG. 23 illustrates a flat laser cut design (prior to shaping and overmolding) of a ‘rectangular’ cell wire frame design.

FIG. 24 illustrates a flat laser cut design (prior to shaping and overmolding) of another ‘honeycomb’ wire frame design.

FIG. 25 illustrates a flat laser cut design (prior to shaping and overmolding) of another ‘honeycomb’ wire frame design, in which the size of the honeycomb cells are larger relative to the ‘honeycomb’ wire frame design of FIG. 24.

FIG. 26 illustrates a flat laser cut design (prior to shaping and overmolding) of a first type of ‘dragonfly’ wire frame design.

FIG. 27 illustrates a flat laser cut design (prior to shaping and overmolding) of a second type of ‘dragonfly’ wire frame design.

FIG. 28 illustrates a flat laser cut design (prior to shaping and overmolding) of a ‘wavy strut wing’ wire frame design.

FIG. 29 illustrates a flat laser cut design (prior to shaping and overmolding) of a ‘diagonal strut wing’ wire frame design.

FIG. 30 illustrates a ‘rectangular’ cell wire frame design within the collapsible impeller body. The flat laser cut design of the ‘rectangular’ cell wire frame is presented next to this image for viewing of the tapering of the metal wire frame away from the trailing edge.

FIG. 31 illustrates a cross-section (Section A-A) from FIG. 30.

### DETAILED DESCRIPTION

Preferred embodiments of the invention will now be described with reference to the accompanying drawings and non-limiting examples.

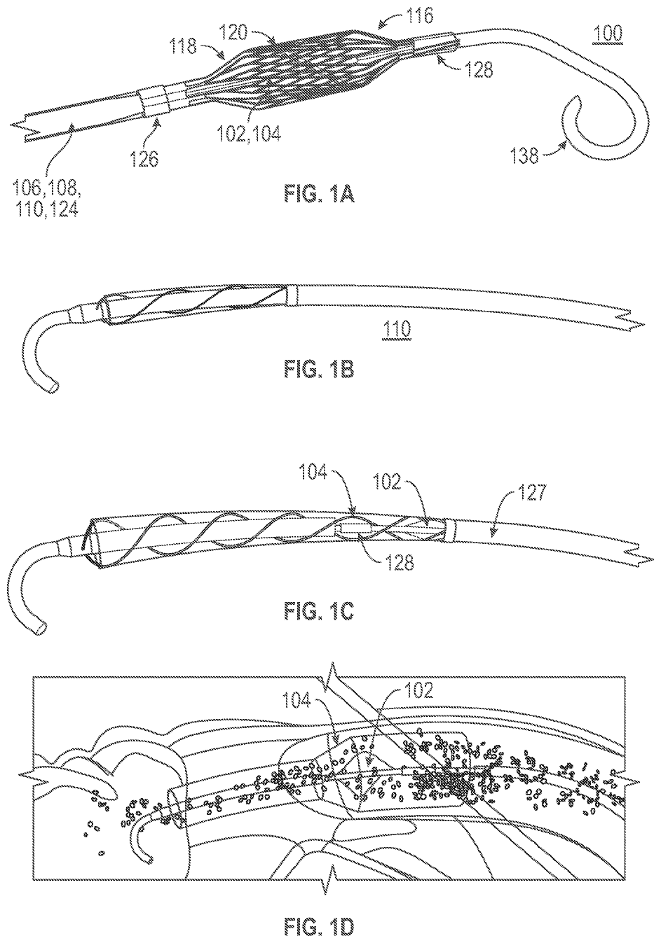

As shown in FIGS. 1A-1D, the anatomy of the blood pump 100 is illustrated. The implantable blood pump 100 may comprise a collapsible impeller 102 mounted in a collapsible impeller cage 104. The drive means or drive cable 106 may be mounted in a housing 108, wherein the housing may be in connection between a first end of a catheter 110 and the collapsible impeller cage 104. The drive means 106 may be adapted for rotating the collapsible impeller 102. There may be a retractable support structure 112 (not shown) adapted to extend from the catheter 110. The extended retractable support structure may be adapted to engage with the wall of the aorta for allowing the implantable blood pump 100 to be secured in the left ventricle of the heart. The collapsible impeller cage 104 may comprise an inlet 116 and an outlet 118. The inlet 116 of the collapsible impeller cage 104 may be adapted to receiving blood from the left ventricle, and wherein the outlet 118 may direct blood in the direction of the atrioventricular valve of the heart. Blood may be directed or moved from the inlet 116 to the outlet 118 by the rotation of the impeller 102. More specifically, the pumping mechanism may be in the form of a miniature axial flow rotary mechanism involving a rotating impeller 102 disposed within the impeller cage 104. The impeller cage 104 may be a collapsible nitinol frame with a flexible polymer coating 120 around the cage 104. The impeller 102 may be mounted to a flexible nitinol frame rotor 122, in which the rotation of the drive cable or drive means 106 may in turn rotate the collapsible rotor 122, which in turn rotates the impeller 102.

The catheter 110 may be hollow in which a drive cable 106 may be arranged in a lumen 124 of the hollow catheter 110. A drive shaft or rotor 122 may be connected to an end of the drive cable to be rotated by the drive cable 106. The ends of the rotor 122 may be in engagement with a proximal bearing 126 and a distal bearing 128. There may be a purge system that may comprise a hole in the hollow catheter, in which the hole may be used for passing fluid for purging and lubrication of the proximal bearing 126 and the distal bearing 128. The purge fluid may pass outside the drive cable 106 in the lumen 124 of the hollow catheter 110 in the direction from the proximal end of the hollow catheter 110 to the distal end of the hollow catheter 110.

As shown in FIG. 1A, the distal bearing 128 may be positioned between the inlet 116 of the impeller cage 104 and the atraumatic tip 138. The atraumatic tip 138 may be disposed distal to the impeller body. The atraumatic tip 138 may be flexible and may have an arcuate configuration such that the atraumatic tip does not injure tissue when contacting tissue, for example, the inner wall or endocardial surface of the ventricle. In some embodiments, the atraumatic tip 138 may have a J-shaped tip. In another embodiment, the distal-most end of the atraumatic tip 138 may include one or more flexible coils, forming a pigtail-shaped tip.

As shown in FIG. 1B, the catheter 110 may be in a compressed state where the impeller 102 and the impeller cage 104 are not exposed. As shown in FIG. 1C, the catheter 110 may be in an expanded state where the outer sheath 127 is moved away from the distal bearing 128 and start expanding the impeller cage 104 and the impeller 102. As shown in FIG. 1D, the catheter 110 is illustrated in a deployed state with the impeller cage 104 and the impeller 102 expanded, showing the blood being pumped by the expanded impeller 102. The collapsibility may be achieved by the use of a nitinol wire frame forming the collapsible impeller cage 104. The collapsible impeller cage 104 can be covered by a membrane, such as a polymer membrane.

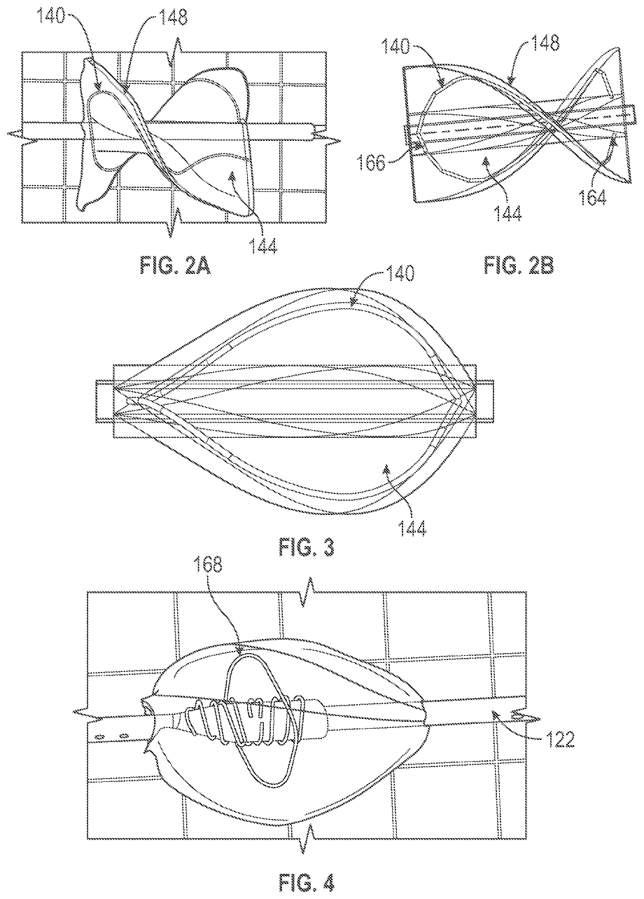

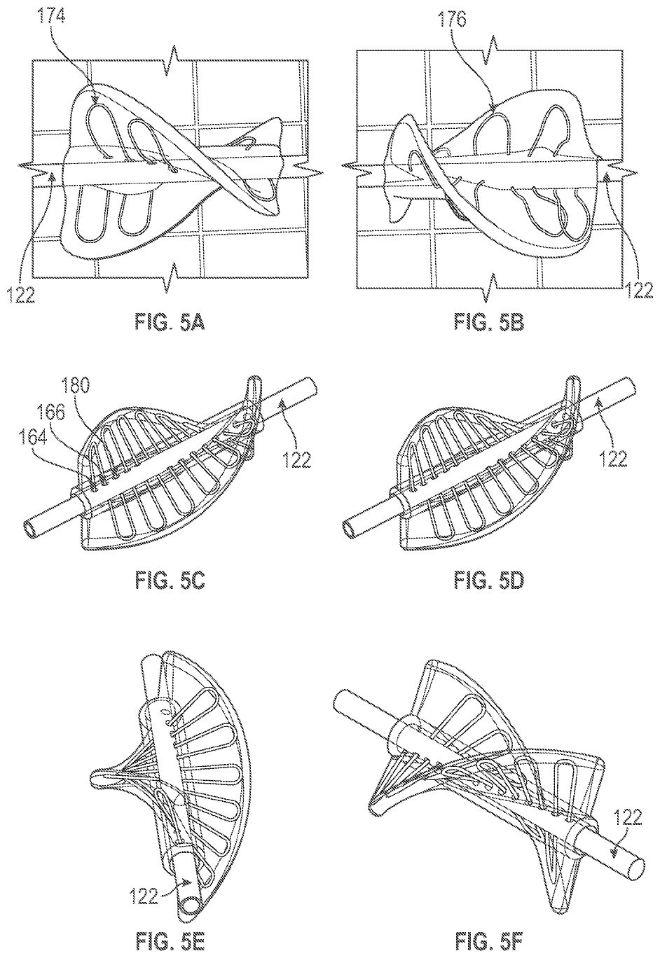

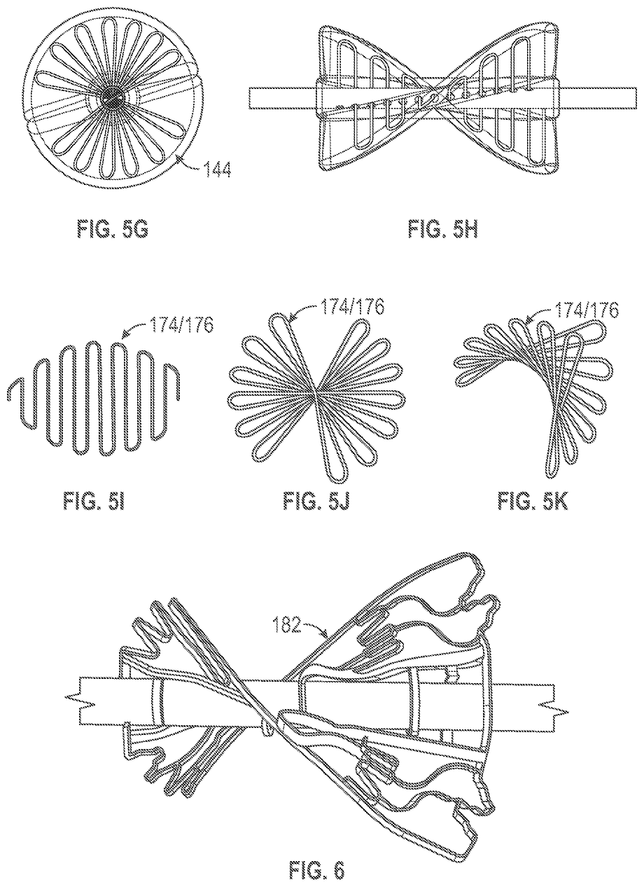

There may also be a membrane for both the impeller cage 104 or the impeller housing 104. As shown in FIGS. 2A, 2B and FIG. 3, the impeller body 144 may be a polymer material and may be translucent such that the wire frame 140 or skeletal shape of the wire frame 140 can be seen within the impeller body 144. An advantage of having a translucent flexible polymer material is so that any wear and tear of the wire frame 140 through usage from collapsing and expanding the impeller body 144 may be identified easily by eye. It may be an advantage to have the wire frame 140 following the contour of the helical shape in the impeller body 144 so that the helical edge 148 is structurally supported. As shown in FIGS. 2A, 2B and FIG. 3, the wire frames 140 supporting the helical edge 148 may have an arcuate portion, wherein a first arcuate wire frame portion 152 is in connection between the first end of a first helical wire frame portion 154 and the first end of a second helical wire frame portion 156, and a second arcuate wire frame portion 158 is in connection between the second end of a first helical wire frame portion 160 and the second end of a second helical wire frame portion 162. The first arcuate wire frame portion 152 may traverse through a first bore 164 of the rotor 122, and the second arcuate wire frame portion 158 may traverse through a second bore 166 of the rotor 122

FIG. 4 illustrates an embodiment where the collapsible impeller 144 may include a petal-shaped wire frame 168 formed of two extensions or two arcuate members attached by or wrapped around the rotor 122. The ‘petal’ wire shape 168 may be configured in such a way that the individual semi-loops form a two bladed impeller, and which is wound in a way that reinforces the rotor 122.

FIG. 5A illustrates a ‘fishbone’ wire frame 174 with reduced loop density and FIG. 5B illustrates another ‘fishbone’ wire frame 176 with reduced loop density while also self-centering. Each of these ‘fishbone’ wire frame embodiments 174/176 may each have a rotor traversing portions that traverses the rotor perpendicularly relative to the longitudinal axis of the rotor 122, which are shown in FIGS. 5C to 5F, and 5H, which are different perspective views of the impeller with the ‘fishbone’ wire frame skeletal shape 174/176. It may also be described as a ‘slalom’ configuration, in which the ‘fishbone’ wire frame skeletal shape 174/176 traverses up through a first bore 164 of the rotor 122 and traverses down through a second bore 166 of the rotor 122 and then traverses up through a third bore 180 of the rotor and so on. The first bore 164 through the rotor 122 may be adjacent to the second bore 166, and the second bore 166 may be adjacent to the third bore 180. FIG. 5G illustrates the top view of the impeller 144 with the ‘fishbone’ wire frame skeletal shape 174/176. FIG. 5H illustrates a side view of the impeller 144 wherein the ‘fishbone’ wire frame follows a ‘slalom’ configuration that reinforces the collapsible rotor.

FIGS. 5I, 5J and 5K may illustrate the wire shape configurations when viewed at different perspectives. For example, the flat wire 174/176 as shown in FIG. 5I may be representative of the wire frame 174/176 as seen in FIG. 5H; while the wire shape 174/176 of FIG. 5J may be representative of the wire frame 174/176 as seen in FIG. 5G; and the wire shape 174/176 of FIG. 5K may be representative of the wire frame 174/176 as seen in FIG. 5F.

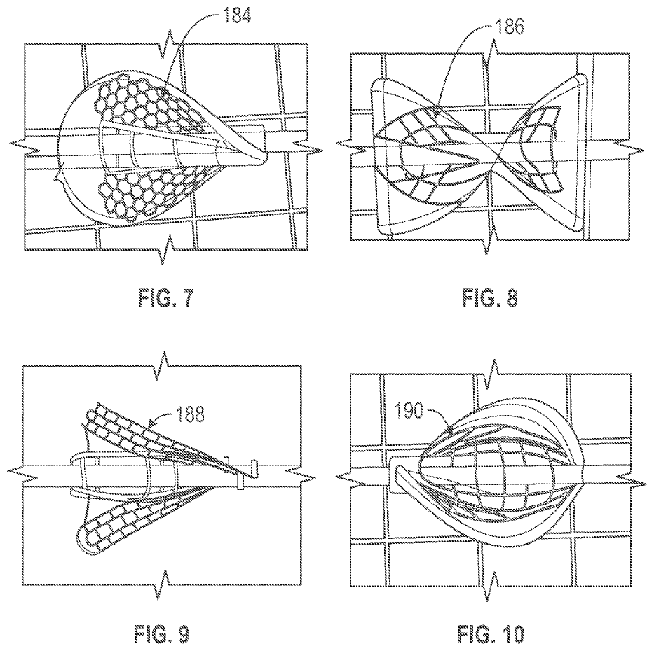

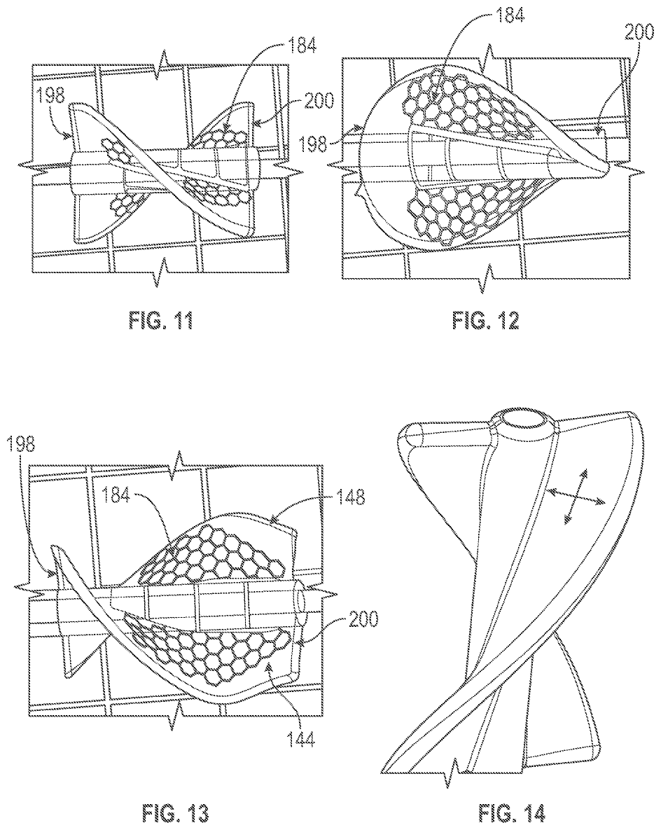

It may be appreciated that other wire frame skeletal shapes such as a ‘wavy strut’ wire frame 182 as depicted in FIG. 6; a ‘honeycomb’ 184 or ‘honeycomb cell’ wire frame 184 as depicted in FIG. 7; a ‘diamond’ 186 or ‘diamond cell’ wire frame 186 as depicted in FIG. 8; different types of ‘dragonfly’ 188 or ‘dragonfly wing’ wire frame(s) 190 as depicted in FIGS. 9 and 10. It may also be appreciated that different impeller shapes with a ‘honeycomb’ wire frame 184 as depicted by FIGS. 11 to 13, the ‘honeycomb’ wire frame 184 may have honeycomb cells present between the rotor 122 and the helical edge or helical curve 148 of the impeller body 144. As shown in FIGS. 11, 12 and 13, there may be less metal or no metal at the corners of the impeller shape or body 144 such as the trailing-edge 198 adjoining to the helical curvature 148 as well as the leading-edge 200 adjoining to the helical curvature 148. This is so that the trailing edge 198 and the leading edge 200 is more easily folded or collapsed as the sheath is advanced from the trailing-edge 198. Once the trailing-edge 198 starts to collapse, it makes it easier for the rest of the impeller body 144 as well as the rotor 122 to also fold. The advantages of the ‘honeycomb’ wire frame skeletal shape 184 is that the honeycomb pattern or hexagonal shapes or cells will have the tendency to resist stretch and compression in the plane that it is oriented, such as shown in FIG. 14, where the arrow direction drawn on the impeller body 144 as a representation. When that plane is heatset or in line into a helix, compound curves can be created. Those compound curves, with their planar stability can become very resistant to deformation of the overall structure. While at the same time, the voids in the plane allow to forcefully overcome the heatset structure temporarily. The displacement of polymer, required to collapse the impeller 144 blades is also aided by the ‘honeycomb’ cellular nature 184 and regularity of the honeycomb structure 184.

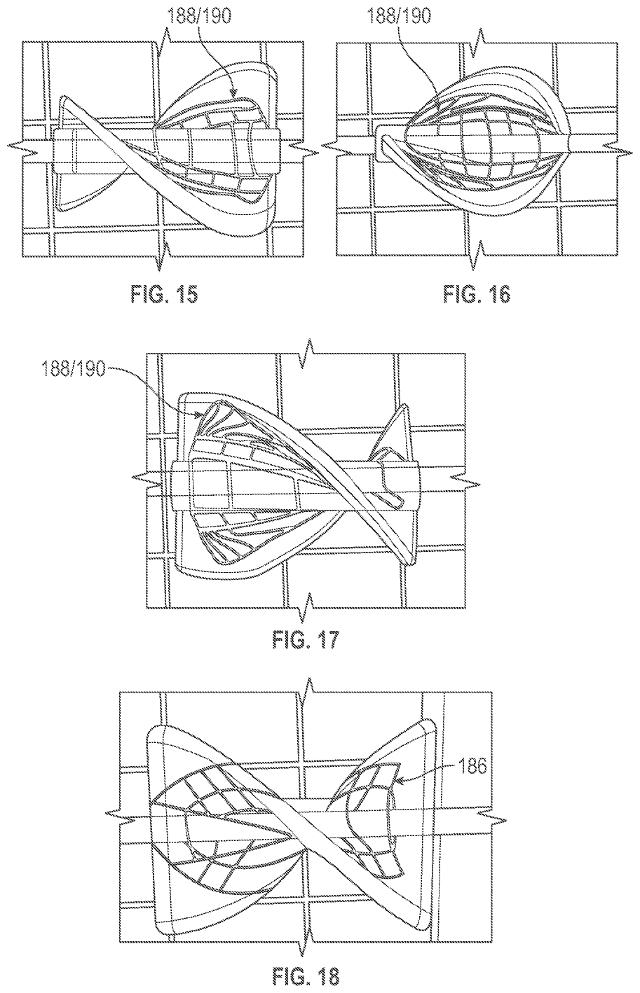

As shown in FIGS. 15 to 17, a ‘dragonfly’ type wire frame 188/190 may be used with the collapsible impeller body 144. Similar to the ‘honeycomb’ wire frame embodiment, there may be less metal or no metal at the corners of the impeller shape such as the trailing-edge 198 adjoining to the helical curvature 148 as well as the leading-edge 200 adjoining to the helical curvature 148. This is so that the trailing edge 198 and the leading edge 200 is more easily folded or collapsed as the sheath is advanced from the trailing-edge 198. Once the trailing-edge 198 starts to collapse, it makes it easier for the rest of the impeller body 144 with the ‘dragonfly’ wire frame skeletal structure 188/190 as well as the rotor 122 to also fold.

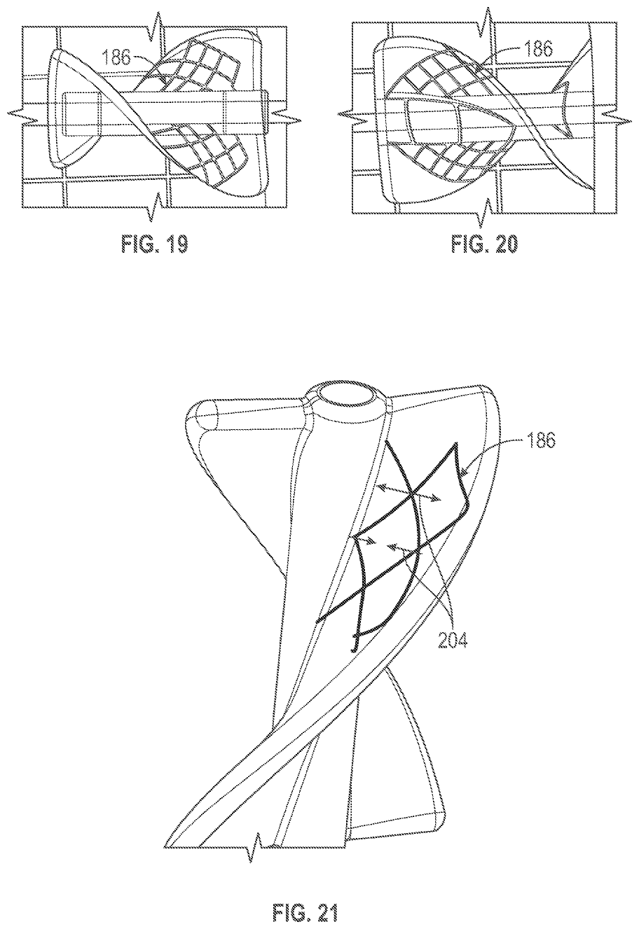

As shown in FIGS. 18, 19 and 20, a ‘diamond’ type wire frame 186 may be used with the collapsible impeller body 144. Similar to the ‘honeycomb’ and the ‘dragonfly’ wire frame embodiment, there may be less metal or no metal at the corners 496 of the impeller shape such as the trailing-edge 198 adjoining to the helical curvature 148 as well as the leading-edge 200 adjoining to the helical curvature 148. This is so that the trailing edge 198 and the leading edge 200 is more easily folded or collapsed as the sheath is advanced from the trailing-edge 198. Once the trailing-edge 198 starts to collapse, it makes it easier for the rest of the impeller body 144 with the ‘diamond’ wire frame skeletal structure 186 as well as the rotor 122 to also fold. Expanded diamond structures 186 are advantageously used for collapsing in medical stents and architecturally in expanded steel. The expanded diamond structures 186 are an economical way to make planar shapes with less material. Diamond structures in a plane can have differing resistance to stretch and compression along different axes. The diamonds can be oriented within the helical plane to utilize compressibility or expandability where necessary to aide in the collapse of the helically planar blades. The representation is shown in FIG. 21 where the outline of the diamond shapes or diamond cells depicted on the impeller body 144 represents the ‘diamond’ cell wire frame 186 and the two headed arrows 204 show the compressibility and expandability of this particular wire frame structure 186. Long struts comprised of numerous edges of individual diamonds or diamond cells could be utilized to create strength by directly spanning longer distances, for a given application (which is a trait less available in the ‘honeycomb’ wire frame embodiment). The displacement of polymer required to collapse the blades may also be aided by the cellular nature and regularity of the ‘diamond’ structure 186.

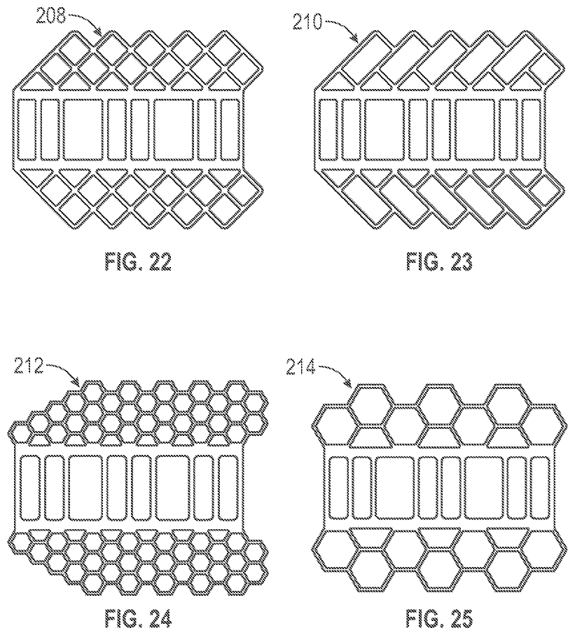

Other wire frame designs are shown in the flat laser cut representations as shown in FIGS. 22 to 29. More specifically, FIG. 22 depicts a flat laser cut representation of a ‘diamond’ cell 208 as shown; FIG. 23 depicts a flat laser cut representation of a ‘rectangular’ cell 210 as shown; FIG. 24 depicts a flat laser cut representation of a ‘honeycomb’ cell 212 as shown, which may be embedded or within the impeller body as shown in FIG. 11, 12 or 13. FIG. 25 depicts a larger ‘honeycomb’ cell 214 as shown, in which for comparison, the hexagonal cells of the bigger ‘honeycomb’ wire frame 214 are larger than the hexagonal cells of the smaller ‘honeycomb’ wire frame 212 as shown in FIG. 24. A possible advantage of using a larger ‘honeycomb’ wire frame 214 is that less metal is used in the construction of this wire frame 214 compared to a smaller ‘honeycomb’ wire frame 212.

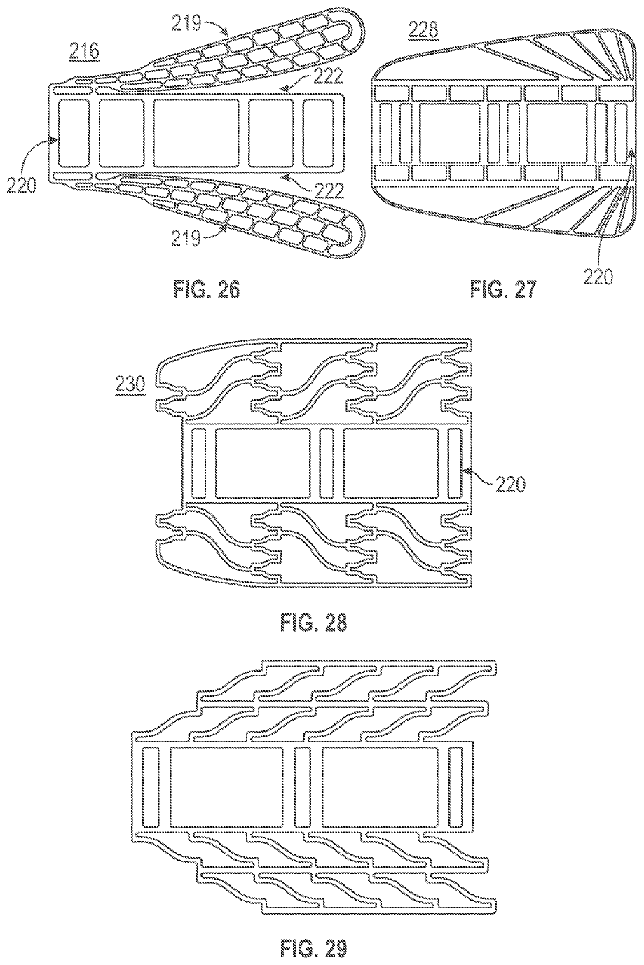

FIG. 26 depicts a ‘dragonfly wing’ wire frame 216 as shown. The ‘wings’ 219 may curve away from the rotor engagement means 220. Each curved ‘wings’ 219 will render gaps 222 between the wing 219 and the rotor engagement means 220. The gaps 222 have no metal and so may allow more flexibility in those areas of the impeller body 144. The increased flexibility in those areas from this wire frame design 216 of the impeller body 144 to be more easily folded or collapsed as the sheath is advanced from the trailing-edge 198. Once the trailing-edge 198 starts to collapse, it makes it easier for the rest of the impeller body with the ‘dragonfly wing’ wire frame skeletal structure 216 as well as the rotor 122 to also fold. The ‘wings’ 219 may have reinforcement by slanted wing struts, which may be offset relative to the adjacent layer so that the slanted wing strut may support the middle of the ‘wing cell’ in the layer above or below. The style at the ‘wing’ may be similar to a ‘brick wall’ configuration. FIG. 27 depicts another type of a ‘dragonfly wing’ wire frame 228 as shown. This ‘wing’ design 228 may have more rotor engagement means reinforcement 220. This may provide more firm and secure attachment of the wire frame to the collapsible rotor 122 and/or provide more firm and secure attachment of the wire frame design 228 to the rotor engagement means 220. There may also be slanted wing struts from the edge of the wire frame 228 to the rotor engagement means reinforcement 220. The slanted wing struts may be for providing additional support to the edge of the ‘dragonfly wing’ wire frame 228.

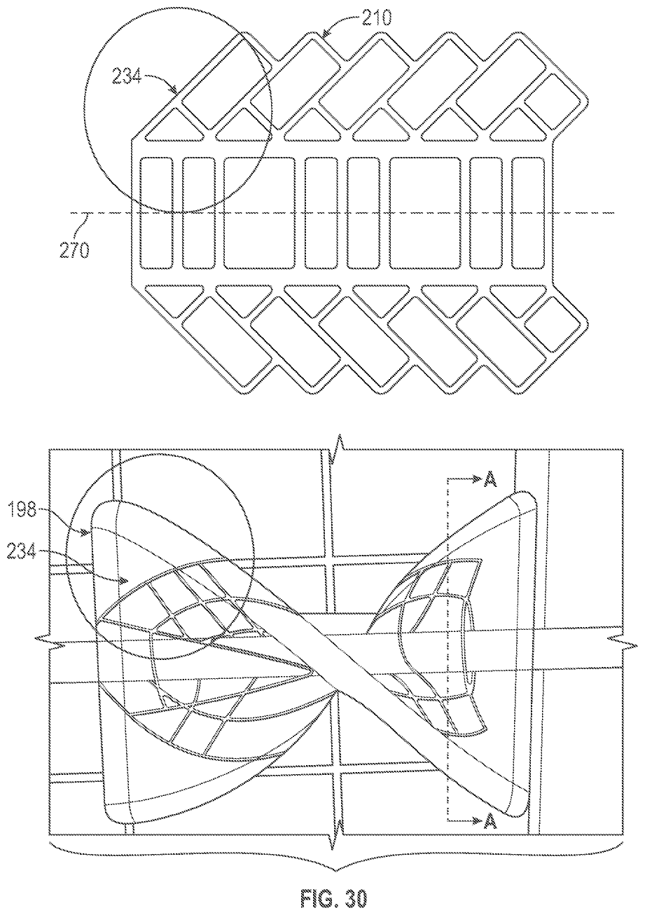

FIG. 28 depicts a ‘wavy strut wing’ wire frame 230 as shown. FIG. 29 depicts a ‘diagonal strut wing’ wire frame 232 as shown. The ‘wavy strut wing’ or ‘diagonal strut wing’ design 232 may have a similar rotor engagement means 220 reinforcements 220 to the ‘dragonfly wing’ wire frame as shown in FIG. 26. There may be ‘wavy’ strut supports for the ‘wavy strut wing’ wire frame 230, and there may be ‘wavy’ strut supports for the ‘diagonal strut wing’ wire frame 232. The ‘wavy’ strut supports provides structure to the impeller body 144. It also assists in reducing stress at the wire frame by spreading out the force to the flexible impeller body 144 caused by conveying liquid or blood when in operation. The tapering 234 at the trailing edge 198, as circled in FIG. 30 shows that there may be less metal or no metal at the corners of the impeller shape 144 such as the trailing-edge 198 adjoining to the helical curvature 148 as well as the leading-edge 200 adjoining to the helical curvature 148. This is so that the trailing edge 198 and the leading edge 200 is more easily folded or collapsed as the sheath is advanced from the trailing-edge 198. The tapering 234 can be seen in flat laser cut wire frame designs FIGS. 22, 23, 24, 25, 26, 27, and 29. In FIG. 30, the example of the flat laser cut ‘rectangular’ cell wire frame design 210 is shown, while the ‘diamond’ cell wire frame design 208 is shown within the translucent impeller body 144 with the tapering 234 circled to show that the corners 196 of impeller shape may have no metal there. The structural frame 140, such as the diamond cell wire frame 210, can display symmetry about an axis, such as a longitudinal axis 270. In an example, the longitudinal axis 270 can assume a parallel orientation with a longitudinal axis of the rotor 122, such as the longitudinal axis 270 can coincide with the longitudinal axis of the rotor 122.

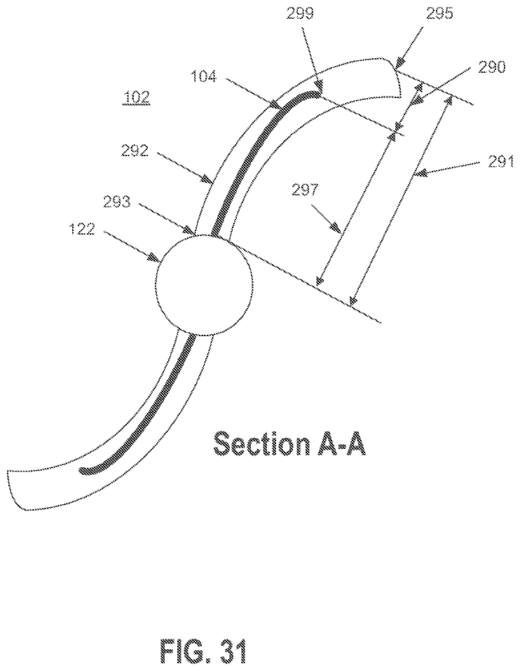

FIG. 31 illustrates a cross-section (Section A-A) of the collapsible impeller 102 from FIG. 30, such as a shown in a plane perpendicular to the longitudinal axis of the rotor 122. The blade of a collapsible impeller 102 can include a structural frame 104 composed of a second material, such as nitinol, embedded in a blade body 292 composed of a first material, such as an elastically deformable polymer. A first material blade edge length 291 can define a distance from a blade attachment location 293 to an outer edge of the first material 295, such as in a plane perpendicular to the rotor 122. A second material blade edge length 297 can define a distance from the blade attachment location 293 to an outer edge of the second material 299, such as in the same plane used to define the first material blade edge length 291. An impeller blade gap 290 can include a distance, such as a difference between the first material blade edge length 291 and the second material blade edge length 297.

The impeller blade gap 290 can be selectable by a user, such as to adjust a stiffness of the blade of the collapsible impeller 102. In an example, a collapsible impeller 102 with a cylindrical shape, such as the radius of the rotor 122 is constant, can have a first material blade edge length 291 that is constant. Where the second material blade edge length 297 can vary along the longitudinal axis of the rotor 122, such as second material blade edge length 297 defined by the outer edge of the frame 210 with respect to the longitudinal axis 270 at different locations along the longitudinal axis 270 shown in FIG. 30, the impeller blade gap 290 can also vary along the longitudinal axis 270.

The impeller blade gap 290 can vary with position along the rotor 122. For example, at a first position on the rotor 122, a plane perpendicular to the longitudinal axis 270 of the rotor 122 can define a first impeller blade gap. At a second position on the rotor 122 different from the first position, a plane perpendicular to the longitudinal axis 270 of the rotor 122 can define a second impeller blade gap, such as different from the first impeller blade gap.

Although the invention has been described with reference to specific examples, it will be appreciated by those skilled in the art that the invention may be embodied in many other forms, in keeping with the broad principles of the invention described herein.

The present invention and the described preferred embodiments specifically include at least one feature that is industrial applicable.

### Various Notes

The above description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventors also contemplate examples in which only those elements shown or described are provided. Moreover, the present inventors also contemplate examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Geometric terms, such as “parallel”, “perpendicular”, “round”, or “square”, are not intended to require absolute mathematical precision, unless the context indicates otherwise. Instead, such geometric terms allow for variations due to manufacturing or equivalent functions. For example, if an element is described as “round” or “generally round,” a component that is not precisely circular (e.g., one that is slightly oblong or is a many-sided polygon) is still encompassed by this description.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1. A radially compressible and expandable rotor for a blood pump, the rotor comprising:
at least one impeller blade having a flexible blade body, wherein the at least one impeller blade includes an elastically deformable first material that is supported by a relatively stiffer second material of a structural frame encapsulated within the blade body, the relatively stiffer second material of the structural frame adapted to give a supporting structure to the impeller blade body, wherein when expanded, the structural frame has a predetermined expanded shape; and
wherein the second material of the structural frame has a shape with a symmetry along a longitudinal axis defined by the rotor.

2. The rotor according to claim 1, wherein the structural frame is attached to an outer surface of a longitudinally elongate rotor hub of the rotor extending along the longitudinal axis.

3. The rotor according to claim 1, wherein the structural frame comprises one or more rotor hub traversing portions, wherein each of the rotor hub traversing portions traverses the rotor hub perpendicularly relative to the longitudinal axis.

4. The rotor according to claim 3, wherein the rotor hub has one or more bores extending into the rotor hub perpendicular to the longitudinal axis.

5. The rotor according to claim 4, wherein the one or more bores comprises:
a first bore, adapted for receiving a first rotor hub traversing portion;
a second bore adapted for receiving a second rotor hub traversing portion, wherein the second bore is both longitudinally offset from the first bore along the longitudinal axis and circumferentially offset from the first bore.

6. The rotor according to claim 5, wherein the structural frame includes an arcuate portion positioned between the first rotor hub traversing portion and a second rotor hub traversing portion.

7. The rotor according to claim 4, wherein the structural frame comprises one or more rotor hub traversing coiled portions, wherein when the structural frame is attached to the rotor hub, each of the one or more coiled portions is respectively coiled between a wall of a respective one of one or more bores and an outer surface of the rotor hub.

8. The rotor according to claim 1, wherein the structural frame is a ‘petal’ skeletal shape.

9. The rotor according to claim 1, wherein the structural frame is a ‘fishbone’ skeletal shape.

10. The rotor according to claim 1, wherein the structural frame comprises a skeletal shape that includes at least one of: a ‘honeycomb’ cell, a ‘diamond’ cell, a ‘rectangular’ cell, a ‘dragonfly’ wing, a ‘wavy’ strut wing, or a ‘diagonal’ strut wing.

11. The rotor according to claim 1. wherein the structural frame is configured to be self-centering within the flexible blade body, when expanded.

12. The rotor according to claim 1, wherein the structural frame is made from a single piece of material.

13. The rotor according to claim 2, wherein the structural frame comprises a rotor engagement frame that is adapted to secure the structural frame to the rotor hub, wherein the rotor engagement frame has an upper frame and a lower frame, wherein the upper frame is parallel to the lower frame, and wherein the upper frame and the lower frame are each parallel to the longitudinal axis.

14. The rotor according to claim 13, comprising a first rotor reinforcement member that is in connection between the upper frame and the lower frame, wherein the rotor reinforcement member comprises a rotor hub engagement portion located between the upper and lower frames and adapted to be secured to a first portion of the outer surface of the rotor hub.

15. The rotor according to claim 14, comprising a second rotor reinforcement member that is adapted to be secured to a second portion of the outer surface of the rotor hub, wherein the secured first portion and the secured second portion of the outer surface of the rotor hub are diametrically opposed from each other across the longitudinal axis.

16. The rotor according to claim 1, comprising an impeller blade gap, defined as a difference between the first material blade edge length and a second material blade edge length, wherein the impeller blade gap is selectable to adjust a stiffness of the at least one impeller blade,
wherein the at least one impeller blade extends from an elongate rotor hub at a blade attachment location,
wherein the first material blade edge length defines a distance from the blade attachment location to an outer edge of the first material of the at least one impeller blade in a plane perpendicular to the longitudinal axis and the second material blade edge length defines a distance from the blade attachment location to an outer edge of the second material of the at least one impeller blade in the plane perpendicular to the longitudinal axis.
